(19) United States
(12) Patent Application Publication
(10) Pub. No.: US 2008/0311638 A1
(43) Pub. Date: Dec. 18, 2008

(54) Shock wave apparatus and methods for ethanol production

(75) Inventors: Tajchai Navapanich, San Diego, CA (US); David Giorgi, Solana Beach, CA (US)

Correspondence Address: CYR & ASSOCIATES, P.A., 605 U.S. Highway 169, Suite 300, Plymouth, MN, 55441, US

(73) Assignee: OptiSwitch Technology Corporation

(21) Appl. No.: 11/904,393

(22) Filed: Sep. 27, 2007

Related U.S. Application Data
(60) Provisional application No. 60/934,782, filed on Jun. 15, 2007.

(51) Int. Cl. C12P 7/10; C12M 1/00

(52) U.S. Cl. 435/165; 435/289.1; 435/303.1

(57) ABSTRACT

Apparatus and methods for ethanol production use shock waves to increase the conversion of starch and/or cellulosic material into sugar. The shock waves may also control bacteria levels in the ethanol production facility. The shock waves may be generated by a shock wave generator that includes a pulsed electric field generator such as a Marx generator, which may have one or more semiconductor switches.

### CROSS-REFERENCE TO RELATED APPLICATIONS

The present application claims the benefit and priority of U.S. provisional patent application 60/934,782 filed on Jun. 15, 2007, the disclosure of which is hereby incorporated by reference in its entirety.

### BACKGROUND OF THE INVENTION

1. Field of the Invention

The present inventions relate to apparatus and methods for ethanol production, and, more particularly, to the apparatus and methods for the production of ethanol with improved efficiencies.

2. Background of the Related Art

The ethanol production facility may use grain as the feed stock to produce ethanol by fermentation of sugar derived from the starch in the grain. Starch is a polysaccharide in which glucose molecules are primarily linked together with alpha(1-4) glycosidic bonds. The ethanol production facility typically includes one or more units configured to solubilize the starch contained within the grain, and to convert the starch into constituent sugars usually using enzymes. The ethanol production facility may include a fermenter configured to ferment the sugar using yeast to produce ethanol, which may include ethanol, butanol and various other alcohols, as well as other chemicals obtainable from the fermentation of sugar. Yeast includes yeast as well as other microorganisms capable of fermenting sugar into ethanol and/or other chemicals. A distillation unit is typically used to capture the ethanol produced by fermentation.

Microcrystalline structures are typically present in the starch that can be resistant to conversion into sugar. For example, these microcrystalline structures typically are resistant to mechanical disruption such as milling and to water penetration, and enzymes do not effectively access the starch contained in the microcrystalline structures to convert the starch into sugar. Yeast metabolize sugar to produce ethanol in the fermenter, and yeast generally cannot metabolize starch including starch bound up in microcrystalline structures. Thus, starch bound up in microcrystalline structures is not converted into sugar and, hence, into ethanol, and may be essentially lost to the ethanol production facility, resulting in inefficiencies in the ethanol production facility.

Alternatively, the ethanol production facility may use cellulosic biomass as feedstock. The ethanol production facility may include one or more units configured to convert the cellulosic material in the cellulosic biomass into sugar using enzymes, and a fermenter configured to ferment the sugar to produce ethanol. The cellulosic material includes cellulose and hemicellulose. Cellulose is a long chain of glucose molecules primarily linked together with beta(1-4) glycosidic bonds and usually embedded in an amorphous matrix of hemicellulose and lignin in the cell walls of the cellulosic biomass. Depending upon the cellulosic biomass, hemicellulose is of varying composition containing branched polymers of, for example, xylose, arabinose, galactose, mannose, and glucose. Hemicellulose is often cross-linked with lignin to create a complex web of bonds which provide structural strength but also challenge degradation. Lignin is a complex polymer of non-sugar organic molecules, which can be cross-linked to each other with a variety of chemical bonds, and is highly resistant to degradation. Lignin restricts the conversion of cellulose and/or hemicellulose into sugar by enzymes. The effect of lignin on the availability of the cellulose and/or hemicellulose cell wall components is thought to be largely a physical restriction, with lignin molecules reducing the surface area available to enzymatic penetration and activity. Thus, cellulosic material that remains bound up with lignin is not converted into sugar by the enzymes, and is lost to the ethanol production facility, resulting in inefficiencies in the ethanol production facility.

Bacteria may also cause inefficiencies in the ethanol production facility. In particular, bacteria can interfere with the conversion of sugar into ethanol by yeast in the fermenter. Typically, the bacteria level in the ethanol production facility is controlled by antibiotics, especially during fermentation. The addition of antibiotics to control the bacterial level could lead to antibiotic resistant strains of bacteria, and may, in the near future, be banned from use. The stillage and various products produced from stillage as well as other byproducts of the ethanol production facility are often used for animal feed and could contain antibiotics. The elimination or reduction of antibiotics in stillage and other byproducts of the ethanol production facility would be of value to animal feed producers.

Thus, there is a need for improved apparatus and methods for the production of ethanol that may reduce the usage of antibiotics and may also have increased efficiency.

### SUMMARY OF THE INVENTION

Apparatus and methods in accordance with the present inventions may resolve many of the needs and shortcomings discussed above and may provide additional improvements and advantages that may be recognized by those of ordinary skill in the art upon study of the present disclosure.

The ethanol production facility apparatus includes a plurality of process units for converting feedstock into ethanol. The process units are in fluid communication to enable a liquid based processing stream to flow among the process units. The ethanol production facility apparatus includes a shock wave generator configured to introduce a shock wave into the liquid based processing stream.

Methods include producing a liquid based processing stream from the feedstock and introducing a shock wave into the liquid based processing stream to condition the liquid based processing stream for ethanol production. The methods may also include processing the liquid based processing stream in a plurality of process units to obtain the ethanol.

Other features and advantages of the invention will become apparent from the following detailed description and from the claims.

### BRIEF DESCRIPTION OF THE FIGURES

FIG. 1 illustrates by schematic diagram an exemplary ethanol production facility apparatus;

FIG. 2 illustrates by schematic diagram another exemplary ethanol production facility apparatus;

FIG. 3 illustrates by schematic diagram exemplary portions of an ethanol production facility apparatus;

FIG. 4 illustrates by schematic diagram another exemplary ethanol production facility apparatus;

FIG. 5 illustrates by schematic diagram exemplary portions of a shock wave generator; and,

FIG. 6 illustrates by schematic diagram exemplary portions of a shock wave generator.

All Figures are illustrated for ease of explanation of basic teachings. The extensions of the Figures with respect to number, position, relationship and dimensions of the parts to form the preferred implementation will be explained or will be within the ordinary skill of the art after the following description has been read and understood. Further, the dimensions and dimensional proportions to conform to specific force, weight, strength, and similar requirements for various applications will likewise be within the ordinary skill of the art after the following description has been read and understood.

Where used in various Figures of the illustrations, the same numerals designate the same or similar parts. Furthermore, when the terms “upper,” “lower,” “right,” “left,” “forward,” “rear,” “first,” “second,” “inside,” “outside,” “front,” “back,” and similar terms are used, the terms should be understood to reference the structure shown in the illustrations and utilized to facilitate describing the illustrations.

### DETAILED DESCRIPTION OF THE INVENTION

The ethanol production facility apparatus includes a plurality of process units configured to produce ethanol from a feedstock. In various aspects, the feedstock may be grain including corn, wheat, and barley or cellulosic biomass or combinations of grain and cellulosic biomass, and the ethanol production facility apparatus may be particularly configured to produce ethanol from a grain feedstock, from a cellulosic biomass feedstock, or from combinations thereof. At least one of the process units is configured to accept the feedstock and to generate a liquid based processing stream The process units are in fluid communication to flow the liquid based processing stream amongst the process units to process the liquid based processing stream into ethanol. The ethanol production facility apparatus includes one or more shock wave generators to apply a shock wave (SW) to the liquid based processing stream at a shock wave location generally within the one or more of the process units to breakdown materials in the liquid based processing stream and/or kill bacteria to control the bacteria level in the liquid based processing stream. One or more shock wave generators may apply a shock wave to the liquid based processing stream as the liquid based processing stream flows between process units to breakdown materials in the liquid based processing stream and/or kill bacteria to control the bacteria level in the liquid based processing stream. In various aspects, the shock wave generator may include a pulsed electric field generator such as a Marx generator, a Marx-PFN generator, as well as other pulsers.

In some aspects, the ethanol production facility apparatus includes the shock wave generator to provide a shock wave configured to cause generally the dissolution of starch microcrystalline structures in order to allow the conversion of the starch in the microcrystalline structures into sugar. In some aspects, the ethanol production facility apparatus may include the shock wave generator configured to cause the disruption of starch from other portions of the grain including fiber, protein, and lipids.

In other aspects, the ethanol production facility apparatus includes the shock wave generator to provide a shock wave configured to enhance the separation of cellulosic material from lignin including other cellular materials in order to allow the conversion of the cellulosic material into sugar.

Sugar includes the saccharides that may be derived from starch as well as the saccharides that may be derived from cellulosic material such as glucose, xylose, mannose, galactose, rhamnose, arabinose, D-pentose sugars, L-sugars, and other saccharides, and combinations thereof, as would be recognized by those of ordinary skill in the art upon study of this disclosure.

In still other aspects, the ethanol production facility apparatus includes the shock wave generator to provide a shock wave configured to kill bacteria in order to control bacteria levels in the ethanol production facility apparatus.

Methods described herein include, in some aspects, providing an ethanol production facility apparatus using grain as feedstock and having a liquid based processing stream, the ethanol production facility apparatus including a shock wave generator, and applying a shock wave to the liquid based processing stream at power and frequency effective to cause generally the dissolution of starch microcrystalline structures using the shock wave generator. In some aspects, the methods may include providing an ethanol production facility apparatus using grain as feedstock and having a liquid based processing stream, the ethanol production facility apparatus including a shock wave generator, and generating shock waves in the liquid based processing stream at pressure and frequency effective to denature starch molecules using the shock wave generator. In some aspects, the methods may include providing an ethanol production facility apparatus using grain as feedstock and having a liquid based processing stream, the ethanol production facility apparatus including a shock wave generator, and generating shock waves in the liquid based processing stream at pressure and frequency effective to cleave starch molecules using the shock wave generator. In some aspects, the methods include providing an ethanol production facility apparatus using grain as feedstock and having a liquid based processing stream, the ethanol production facility apparatus including a shock wave generator, and applying a shock wave to the liquid based processing stream at power and frequency effective to cause the disruption of starch from other portions of the grain including fiber, protein, and lipids using the shock wave generator.

In other aspects, the methods include providing an ethanol production facility apparatus using cellulosic biomass as feedstock and having a liquid based processing stream, the ethanol production facility apparatus including a shock wave generator, and applying a shock wave to the liquid based processing stream at power and frequency effective to cause the disruption of the cellulosic material structures from lignin using the shock wave generator. In some aspects, the shock wave may be effective to break intermolecular and/or intramolecular bonds within and/or between the cellulose, hemicellulose, and/or lignin.

In various aspects, the methods include providing an ethanol production facility apparatus having a liquid based processing stream, the ethanol production facility apparatus including a shock wave generator, and applying a shock wave to the liquid based processing stream at a power and frequency effective to kill bacteria in order to control the bacteria level using the shock wave generator.

The Detailed Description and the Figures illustrate exemplary ethanol production facility apparatus and methods. These illustrated apparati and methods are not meant to limit the scope of coverage but, instead, to assist in understanding the context of the language used in this specification and in the appended claims. Accordingly, the appended claims may encompass variations that differ from the illustrations.

The ethanol production facility apparatus includes the shock wave generator to generate shock waves. The shock wave generator, in some aspects, may be configured as an electro-hydraulic generator where electrodes are submerged in a water-filled housing. The electro-hydraulic generator initiates the shock wave by an electrical discharge between the electrodes. Vaporization of water molecules between the electrodes produces vapor bubbles that grow and rupture resulting in an explosion thus generating the shock wave. The electro-hydraulic generator generates a shock wave with a fast rise time and generates focused energy over a broad area to deliver a large amount of energy. In addition, the shock waves can be further focused through the use of focusing reflectors in an electro-hydraulic generator.

The shock wave generator, in some aspects, may be configured as an electromagnetic generator where opposing metal membranes are connected to electromagnetic coils. When a high current passes through one coil, a strong magnetic field is generated that induces a high current in the opposing membrane and accelerates the metal membrane away from the coil to generate a pressure wave.

In some aspects, the shock wave generator may be configured as a piezoelectric generator form shock waves by the application of high voltage pulses to the piezoelectric crystals, which convert electrical signals into mechanical vibrations. The crystals contract and expand, generating the shock wave. Pulsed electric discharges in a solid dielectric immersed in liquid such as water can create shock waves on the order of 1,000-10,000 MPa.

Organic polymers such as proteins and polysaccharides can be denatured through the application of shock waves with pressures ranging from about 300-600 MPa generally at ambient temperature or with reduced heat input. The application of hydrostatic pressure to starch can cause the gelatinization of starch. At temperatures greater than about 40° C. and pressures greater than about 500 MPa, the susceptibility of starches to amylase digestion is greatly increased.

There are three types of molecular bonds that exist in the starch matrix: covalent, hydrogen, and van der Waals. Of these three, the dissociation energy of the covalent bonds is one to two orders of magnitude higher than the other two. At pressures between 1000-10000 MPa, the covalent bonds can be dissociated and the starch matrix broken down into both amylose and amylopectin functional groups. Accordingly, the shock wave generator 120 may generate shock waves with transient pressures of more than about 1000 MPa, so that the conversion of starch to sugar by enzymes such as alpha-amylase and gluco-amalyse may be achieved generally at ambient temperatures with little or no heat required. The addition of heat to raise the temperature of the liquid based processing stream to about 40° C. may reduce the conversion time and increase the enzyme efficiency.

One or more shock wave generators may be disposed about the ethanol production facility apparatus to generate shock waves configured to cause generally the dissolution of starch microcrystalline structures in order to allow the conversion of the starch in the microcrystalline structures into sugar.

Shock waves can kill bacteria. Most bacteria are killed by shock waves with pressures between about 300-600 MPa at room temperature. Accordingly, the shock wave generator may generate high pressure shock waves to eliminate or reduce the number of bacteria in the liquid based processing stream, which may increase the effectiveness of the yeast during fermentation.

One or more shock wave generators may be variously disposed about the ethanol production facility apparatus to apply shock waves at locations effective to control the bacteria level in the liquid based processing stream. In some aspects, one or more shock wave generators may be disposed about the ethanol production facility apparatus and configured to generate shock waves in the liquid based processing stream at a location effective to control the bacteria level in the fermenter.

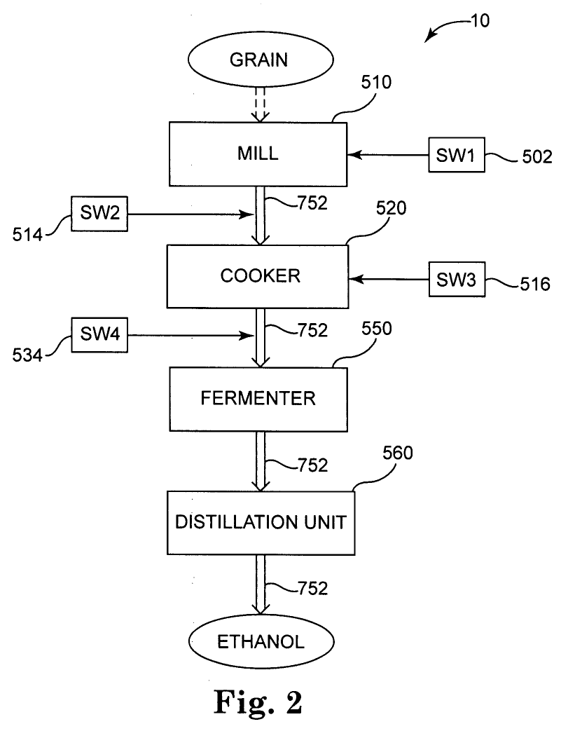

As shown in FIG. 2, an ethanol production facility apparatus 10 configured to use grain as feedstock include a plurality of process units in fluid communication to flow the liquid based processing stream amongst the process units, and the process units are configured to process the liquid based processing stream to convert the starch in the grain feedstock into ethanol. The process units include a mill 510, a cooker 520, a fermenter 550, and a distillation unit 560. Grain may be input into the mill 510, and the mill 510 is usually configured to destroy the structure of the grain by conversion of the grain into grain fragments such as a meal and/or powder in order to allow water including other suitable liquids, such as aqueous-based solvents, to contact the starch contained within the grain and to mix the grain fragments with water to form the liquid based processing stream. The liquid based processing stream may include solvents that are aqueous-based, organic-based, or inorganic-based. Furthermore, the solvents in the liquid based processing stream may span the pH range from acid to basic.

The mill 510, the cooker 520, the fermenter 550, and the distillation unit 560 are in fluid communication so that the liquid based processing stream may flow from the mill 510 to the cooker 520, from the cooker 520 to the fermenter 550, and from the fermenter 550 to the distillation unit 560. The liquid based processing stream is communicated by pipe 752 including channels, ducts, troughs, and other conveyance structures in various implementations. The nature of the liquid based processing stream generally changes from slurry to mash to fermented mash, and, finally, to ethanol and stillage as the liquid based processing stream is communicated through various process units of the ethanol production facility apparatus 10. Various pumps, pipes, valves, vessels, ducts, storage reservoirs, heat exchangers, boilers, process control systems, electrical power systems, and other such apparatus may be provided as part of the ethanol production facility apparatus 10 inter alia to communicate the liquid based processing stream between the mill 510, the cooker 520, the fermenter 550, and the distillation unit 560, and/or as components of the mill 510, the cooker 520, the fermenter 550, the distillation unit 560, and/or other process units. The process units including the mill 510, the cooker 520, the fermenter 550, and the distillation unit 560 are generally configured to separate the starch in the liquid based processing stream from other portions of the grain feedstock, to convert the starch in the liquid based processing stream to sugar, and to ferment the sugar in the liquid based processing stream to produce ethanol.

Starch may be bound up with other portions of the grain in grain fragments, and starch may be bound up in starch microcrystalline structures in the liquid based processing stream. One or more shock wave generators may be disposed about the ethanol production facility apparatus 10 to provide a shock wave configured to cause generally the dissolution of starch microcrystalline structures in order to allow the conversion of the starch into sugar. In various aspects, one or more shock wave generators may be disposed about the ethanol production facility apparatus 10 to provide a shock wave configured to loosen the structure of the grain fragments to enhance solubilization of the starch in the grain fragments. The shock wave generator may generate a shock wave configured to separate the starch from other portions of the grain including fiber, lipid, and protein. Shock waves are effective in killing bacteria in food processing. One or more shock wave generators may be variously disposed about the ethanol production facility apparatus 10 to apply shock waves at locations effective to control the bacteria level in the liquid based processing stream.

The ethanol production facility apparatus 10 may be configured in other ways to use grain as feedstock to produce ethanol. One or more shock wave generators may be disposed about said ethanol production facility apparatus 10 in various ways to cause the dissolution of starch microcrystalline structures, to loosen the structure of grain fragments, and/or to control bacteria levels, as would be recognized by those of ordinary skill in the art upon study of this disclosure.

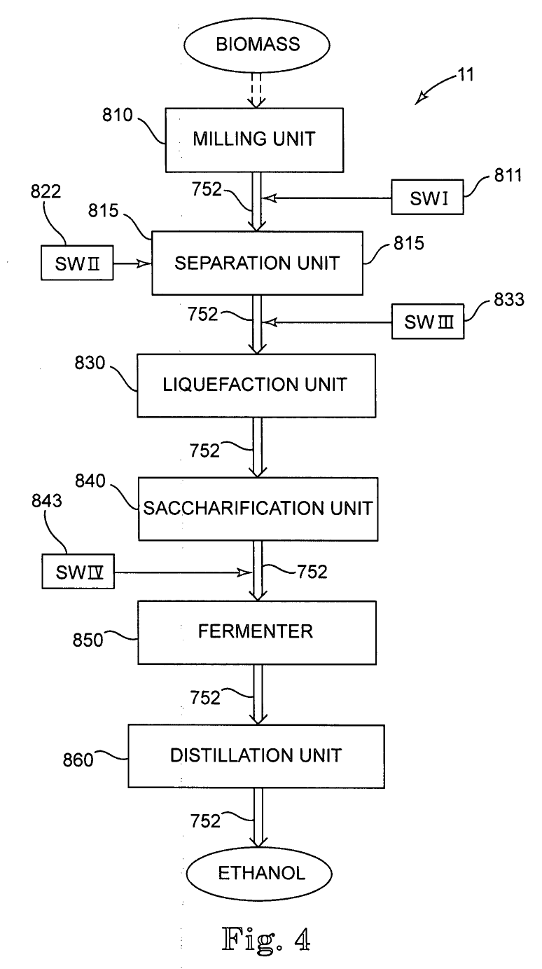

As illustrated in FIG. 4, an ethanol production facility 11 configured to use cellulosic biomass as feedstock for the production of ethanol includes a plurality of process units in fluid communication to convey a liquid based processing stream between the process units, where the process units are configured to convert the cellulosic material in the cellulosic biomass feedstock into ethanol. The process units, in various aspects, may include a mill unit 810, a separation unit 815, a liquefaction unit 830, saccharification unit 840, fermenter 850, and distillation unit 860. Cellulosic biomass, which may be in the form of pressed sugar cane, corn stalks, wood, and other plant materials and combinations of plant materials, may be input into the mill unit 810. In some implementations, cellulosic biomass may be in the form of waste materials from, for example, food processing, industrial processes, and waste water treatment (e.g. sewage sludge). The mill unit 810 may be configured to degrade the cellulosic biomass in size to produce cellulosic biomass fragments and to mix the cellulosic biomass fragments with water, or other aqueous, organic or inorganic based solvents of varying pH, to form the liquid based processing stream.

The mill unit 810, the separation unit 815, the liquefaction unit 830, the saccharification unit 840, the fermenter 850, and the distillation unit 860 are in fluid communication by pipe 752, so that the liquid based processing stream may be communicated from the mill unit 810 to the separation unit 815, from the separation unit 815 to the liquefaction unit 830, from the liquefaction unit 830 to the saccharification unit 840, from the saccharification unit 840 to the fermenter 850, and from the fermenter 850 to the distillation unit 860 in this implementation. Various pumps, pipes, valves, vessels, storage reservoirs, heat exchangers, boilers, process control systems, and other such apparatus may be provided as part of the ethanol production facility apparatus 11, inter alia, to communicate the liquid based processing stream between the mill unit 810, the separation unit 815 the liquefaction unit 830, the saccharification unit 840, the fermenter 850, and the distillation unit 860.

As illustrated in FIG. 4, the process units including the mill unit 810, the separation unit 815 the liquefaction unit 830, the saccharification unit 840, the fermenter 850, and the distillation unit 860 are generally configured to separate the cellulosic material in the liquid based processing stream from other portions of the cellulosic biomass feedstock, to convert the cellulosic material in the liquid based processing stream to sugar, and to ferment the sugar in the liquid based processing stream to produce ethanol.

The cellulosic material may be bound up with other portions of the cellulosic biomass such as lignin. For example, the cellulosic biomass fragments may be composed of one or more plant cells and/or portions of plant cells. The shock waves may cause damage to plant cell walls as well as membranes of internal organelles within the plant cells, leading to easier passage of material into and out of the cellulosic biomass fragments, which may enhance the separation of cellulosic material from lignin contained in the cellulosic biomass fragments. Accordingly, one or more shock wave generators may be disposed within the ethanol production facility apparatus 11 to provide a shock wave configured to cause generally damage to cellulosic biomass fragments in order to enhance the separation of cellulosic material from lignin in the cellulosic biomass fragments.

The use of acids and/or bases to separate cellulosic material from lignin or hydrolize the cellulosic material into sugar may form undesirable byproducts such as acetic acid, formic acid, levulinic acid, phenol, vanillin, furfural and hydroxymethyl furfural (HMF). Such byproducts formed during the hydrolysis pretreatment step are known to be toxic to yeast or to inhibit yeast metabolism thereby reducing the fermentation efficiency of yeast. Furthermore, compounds such as furfural and HMF result from the breakdown of pentoses (e.g., xyloxe) and hexoses (e.g., glucose) thereby reducing the amount of fermentable sugars available for ethanol production. The application of shock waves to the liquid based processing stream by one or more shock wave generators may reduce or eliminate the use of acids and/or bases, and, hence, reduce or eliminate the byproducts. The elimination of the byproducts may increase the efficiency of ethanol production.

For example, in various aspects, one or more shock wave generators may be disposed about the ethanol production facility apparatus 11 to provide the shock wave to the liquid based processing stream in order to enhance the separation of cellulosic material from lignin. In various aspects, one or more shock wave generators may be disposed about the ethanol production facility apparatus 11 to provide shock wave to the liquid based processing stream configured to hydrolyze the cellulosic material into sugar. In various aspects, one or more shock wave generators may be disposed about the ethanol production facility apparatus 11 to provide shock wave to the liquid based processing stream to kill bacteria in order to control the bacteria level in the liquid based processing stream.

The ethanol production facility apparatus 11 may be configured in other ways to use cellulosic biomass as feedstock to produce ethanol. One or more shock wave generators may be disposed about said ethanol production facility apparatus 11 in various ways to control bacteria levels and/or to enhance the separation of cellulosic material from lignin, as would be recognized by a person of ordinary skill in the art upon study of this disclosure.

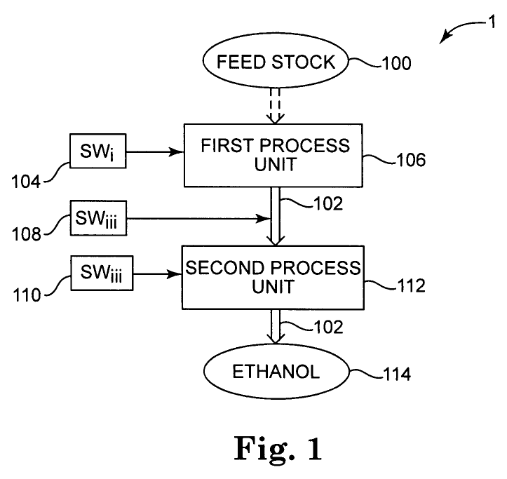

FIG. 1 illustrates a general ethanol production facility apparatus 1 that includes a plurality of process units. In this illustration, the process units include a first process unit 106 and a second process unit 112 configured to produce ethanol 114 from a feedstock 100. The first process unit 106 accepts a feedstock 100 and generates the liquid based processing stream that includes at least portions of the feedstock 100. The first process unit 106 and the second process unit 112 are in fluid communication to communicate a liquid based processing stream by a section of pipe 102 from the first process unit 106 to the second process unit 112. Ethanol 114 is output from the second process unit 112 in this illustration.

In FIG. 1, the shock wave generator 104 applies a shock wave to the liquid based processing stream at a shock wave location generally within the first process unit 106 to break down materials in the liquid based processing stream and/or kill bacteria to control the bacteria level in the liquid based processing stream. Shock wave generator 108 applies a shock wave to the liquid based processing stream at a shock wave location in the pipe 102 between the first process unit 106 and the second process unit 112 to break down materials in the liquid based processing stream and/or kill bacteria to control the bacteria level in the liquid based processing stream. The shock wave generator 110 in the illustration applies a shock wave to the liquid based processing stream at a shock wave location generally within the second process unit 112 to break down materials in the liquid based processing stream and/or kill bacteria to control the bacteria level in the liquid based processing stream.

The ethanol production facility apparatus 10 (FIG. 2), which includes one or more shock wave generators to provide shock waves at one or more locations, is now described in more detail and may be modified in various ways examples of which are also described. The ethanol production facility apparatus 10, as illustrated, includes a plurality of process units including mill 510, cooker 520, fermenter 550, and distillation unit 560 in fluid communication by pipes 752. The arrows generally denote the flow of materials including the liquid based processing stream through the pipes 752 in the ethanol production facility apparatus 10.

As illustrated in FIG. 2, the mill 510 accepts grain as feedstock. The mill 510 may include a hammer-mill and various grinders, and other milling machines to convert the grain into grain fragments such as a meal and/or powder in order to allow water to contact the starch contained within the grain. The mill 510 mixes the grain fragments with water to form the liquid based processing stream. The liquid based processing stream is communicated from the mill 510 to the cooker 520.

The cooker 520 includes one or more vessels configured to heat the liquid based processing stream communicated from the mill 510 along with enzymes such as alpha-amylase in order to solubilize and liquefy the starch in the grain fragments in liquid based processing stream. This may be referred to as gelatinization and liquefaction, respectively. The cooker 520 may be configured to implement various cooking processes such as jet cooking, which may occur at temperatures in excess of 100° C. and at pressures of several atmospheres. The heat and/or pressure in the cooker 520 may cause water molecules to be adsorbed or absorbed by the starch, which may cause the starch molecules to expand, weaken the structure of the starch, and solublilize the starch molecules. The enzymes such as alpha-amylase generally cleave the long polysaccharide chains of the starch molecules into sugar chains such as maltodextrins and oligosaccharides to liquefy the starch, and may also weaken the structure of the starch to solubilize the starch molecules.

Shock wave generators may be disposed about the ethanol production facility apparatus 10, as illustrated in FIG. 2, to apply shock waves to the liquid based processing stream. The shock wave generators may generate shock wave configured to facilitate breakdown of materials in the liquid based processing stream including the dissolution of starch microcrystalline structures in order to allow the conversion of the starch in the microcrystalline structures into sugar. The shock wave generators apply a shock wave at locations within the ethanol production facility apparatus 10 as indicated.

As illustrated in FIG. 2, one or more shock wave generators 502 may apply a shock wave to the liquid based processing stream at a shock wave location generally within the mill 510. This applied shock wave loosens the structure of the grain fragments to generally loosen and/or separate the starch from other portions of the grain, which enhances solubilization of the starch as well as the conversion of starch into sugar in later processes. One or more shock wave generators 514 may apply a shock wave to the liquid based processing stream within the pipe 752 at a shock wave location between mill 510 and cooker 520. The applied shock wave loosens the structure of the grain fragments produced by mill 510 to generally separate the starch from other portions of the grain.

One or more shock wave generators 516 may apply a shock wave to the liquid based processing stream generally within the cooker 520 to cause generally dissolution of starch microcrystalline structures in order to produce a more complete solubilization of starch, as illustrated in FIG. 2. The applied shock wave allows the cooker 520 to use lower temperatures and/or shorter processing times, which may increase the energy efficiency of the ethanol production facility apparatus 10.

The liquid based processing stream is communicated from the cooker 520 to the fermenter 550 through pipe 752 in the illustration of FIG. 2. Saccharification and fermentation are combined in the fermenter 550 in this implementation. Other implementations of the ethanol production facility apparatus 10 may include a first unit and a second unit configured for saccharification and fermentation, respectively. Thus, in various implementations, saccharification may either be followed by fermentation, or, as illustrated, saccharification may be concurrent with fermentation. As illustrated in FIG. 2, the fermenter 550 is configured to use enzymes to convert the sugar chains in the liquid based processing stream into sugars such as glucose that are fermentable, and yeast to ferment the sugars to produce ethanol

One or more shock wave generators 534 may apply the shock wave to the liquid based processing stream within the pipe 752 at a shock wave location between cooker 520 and fermenter 550 in the implementation illustrated in FIG. 2. This applied shock wave may generally cause dissolution of starch microcrystalline structures to produce a more complete solubilization of starch in the liquid based processing stream. This applied shock wave may be configured to kill bacteria in the liquid based processing stream to control the bacteria level in fermenter 550. By controlling the bacterial level in the fermenter 550, the bacterial interference with the conversion of sugar into ethanol by yeast during fermentation, with corresponding inefficiencies in the ethanol production facility apparatus 10, is reduced. In implementations of the ethanol production facility 10 with a first process unit and a second process unit, the shock wave may be applied to the liquid based processing stream between the first process unit and the second process unit to control bacterial levels in the second process unit (fermentation). The provision of shock waves at other locations in the ethanol production facility apparatus 10 may also control bacteria levels in the ethanol production facility apparatus 10 to reduce or eliminate the use of antibiotics in the ethanol production facility apparatus 10. Shock waves may be provided at various locations in cellulosic biomass based ethanol production facility apparatus 10 to control bacterial levels.

The liquid based processing stream is communicated from the fermenter 550 to the distillation unit 560 in FIG. 2. The distillation unit 560 is configured to capture the ethanol produced during fermentation from the liquid based processing stream by distillation. In various implementations, the distillation unit 560 may be a configured as a still, distillation column, fractionation column, absorption column, adsorption column, or suchlike to capture the ethanol from the liquid based processing stream. Stillage is the remnant of the liquid based processing stream after the distillation unit 560 captures the ethanol. The distillation unit 560 may also include various units to strip water from the ethanol captured from the liquid based processing stream in order to produce essentially pure ethanol. Stillage, which generally consists of various unfermented materials and waste yeast, may be recovered.

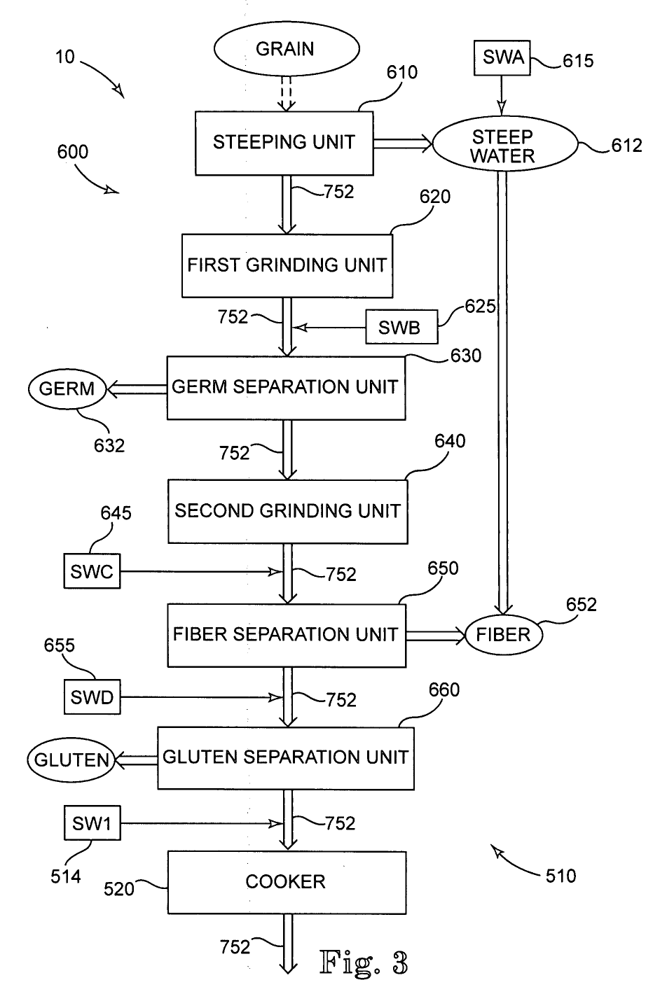

Shock wave generators may be employed in various ways in the mill 510 to apply shock waves to the liquid based processing stream to generally separate the starch from other portions of the grain in order to increase the availability of starch for conversion into sugar. For example, the mill 510 may be implemented as a wet mill 600 (FIG. 3) having a liquid based processing stream. The wet mill 600, as illustrated, includes steeping unit 610, first grinding unit 620, germ separation unit 630, second grinding unit 640, fiber separation unit 650, and gluten separation unit 660 in fluid communication by pipes 752. The steeping unit 610 may be configured to receive generally dry grain and to steep the grain in water thereby forming the liquid based processing stream. One or more shock wave generators 615 may apply shock wave to the steep water 612 produced from steeping unit 610. This applied shock wave causes degradation or denaturization of nucleic acids and protein in the steep water 612. In some implementations, one or more shock wave generators may apply shock waves to the liquid based processing stream in the steeping unit 610.

One or more shock wave generators 625 may apply shock waves to the liquid based processing stream within the pipe 752 at a shock wave location between first grinding unit 620 and germ separation unit 630. The applied shock waves result in enhanced separation of germ in germ separation unit 630, and in enhanced separation of fiber from starch and gluten in the fiber separation unit 650 to increase the availability of the starch for conversion into sugar.

One or more shock wave generators 645 may apply shock waves to the liquid based processing stream within the pipe 752 at a shock wave location between second grinding unit 640 and fiber separation unit 650, as illustrated in FIG. 3. Additionally or alternatively, one or more shock wave generators 655 may apply shock waves to the liquid based processing stream within the pipe 752 at a shock wave location following the fiber separation unit 650 and prior to the gluten separation unit 660 to enhance separation of starch and gluten in order to increase the availability of starch for conversion into sugar.

The ethanol production facility apparatus 11 (FIG. 4) for the production of ethanol from cellulosic biomass is now described in more detail, and may be modified in various ways, examples of which are also described. In this implementation, the ethanol production facility apparatus 11 includes process units configured as milling unit 810, separation unit 815, liquefaction unit 830, fermenter 850, and distillation unit 860 in fluid communication by pipes 752 to communicate the liquid based processing stream. Cellulosic biomass is input into the milling unit 810. Milling unit 810 degrades the cellulosic biomass in size to form cellulosic biomass fragments. Mill unit 810 may include various chippers, grinders, hammer mills, and suchlike configured to degrade the cellulosic biomass in size and otherwise disrupt the structure of the cellulosic biomass to produce cellulosic biomass fragments. The cellulosic biomass fragments may be mixed with water or combinations of aqueous, organic, and inorganic solvents of varying pH, to form the liquid based processing stream. The liquid based processing stream containing the cellulosic biomass fragments may be communicated from milling unit 810 into separation unit 815. Separation unit 815 separates the cellulosic material in the cellulosic biomass fragments from lignin in the cellulosic biomass fragments. The separation unit 815 is configured as one or more vessels in which various chemicals such as acids, bases, solvents, physical forces such as elevated temperatures and/or pressures, and mechanical forces such as stirring and agitation may be applied to the liquid based processing stream to separate the cellulosic material from lignin and other materials. The lignin may be removed from the liquid based processing stream, and the liquid based processing stream containing cellulose and hemicellulose may be communicated by pipe 752 from separation unit 815 to liquefaction unit 830 and, thence, to saccharification unit 840, which reduce the cellulosic material to constituent sugars that may be fermentable by the application of enzymes and heat. The constituent sugars may then be fermented into ethanol by yeast.

The liquid based processing stream is communicated from saccharification unit 840 to the fermenter 850. The fermenter 850 uses yeast to ferment the sugar in order to produce ethanol. The ethanol may be recovered from the liquid based processing stream by distillation unit 860.

As illustrated in FIG. 4, one or more shock wave generators 811 may apply shock waves to the liquid based processing stream within pipe 752 at a shock wave location prior to separation unit 815 to enhance the separation of cellulosic material in the cellulosic biomass fragments from lignin in the cellulosic biomass fragments. Separation may include separation of the cellulosic material from the lignin as well as otherwise increasing the accessibility of the cellulosic material to enzymes. One or more shock wave generators 822 may apply a shock wave to the liquid based processing stream at a shock wave location generally within separation unit 815 to enhance the separation of cellulosic material in the cellulosic biomass fragments from lignin in the cellulosic biomass fragments. One or more shock wave generators 833 may apply a shock wave to the liquid based processing stream within pipe 752 at a shock wave location between separation unit 815 and liquefaction unit 830 to enhance the separation of cellulosic material in the cellulosic biomass fragments from lignin in the cellulosic biomass fragments. Application of shock waves at shock wave location 811, shock wave location 822, and/or shock wave location 833 may enhance the separation of cellulosic material in the cellulosic biomass fragments from lignin in the cellulosic biomass fragments.

One or more shock wave generators 843 may apply shock waves to the liquid based processing stream within pipe 752 at a shock wave location between the saccharification unit 840 and the fermenter 850, as illustrated in FIG. 4. These shock waves may be configured to kill bacteria in the liquid based processing stream to control the bacteria level in the fermenter 850 in order to prevent bacterial interference with fermentation. In other implementations, one or more shock wave generators may apply shock waves configured to kill bacteria at various locations in the ethanol production facility apparatus 11 in order to control the bacteria level generally proximate those locations.

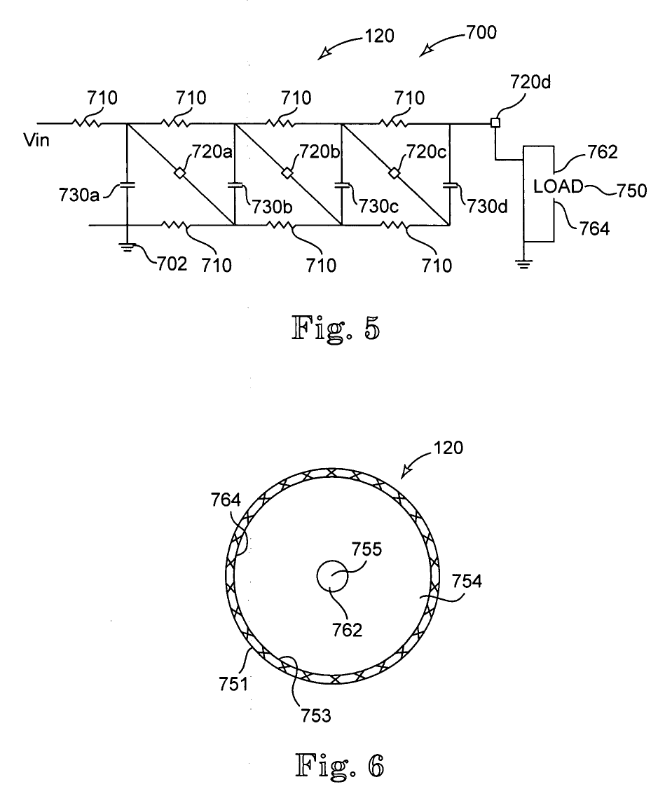

The shock wave generator 120 including the electro-hydraulic system, the electromagnetic generator, and the piezoelectric generator may include a pulsed electric field generator 700 such as a Marx Generator, a Marx-PFN generator, as well as other pulsers to drive the generation of shock waves. An illustration of the shock wave generator configured, in part, as a pulsed electric field generator 700 is illustrated in FIG. 5. The pulsed electric field generator 700 illustrated in FIG. 5 includes four stages, but various other implementations may include fewer or more stages. In this implementation, capacitors 730, which are shown as capacitors 730a, 730b, 730c, 730d, are charged in parallel from voltage source Vᵢₙ. The capacitors 730a, 730b, 730c, 730d may then be discharged in series through load 750 so that the voltage passing through the load 750 is the cumulative voltage discharged by capacitors 730a, 730b, 730c, 730d. Resistors 710 are also included for charging but these might be inductors as well.

The discharge of the capacitors 730a, 730b, 730c, 730d into the load 750 may be controlled by switches 720 illustrated as switches 720a, 720b, 720c, 720d in FIG. 5. The switches 720a, 720b, 720c, 720d may be any of various types of semiconductor switches such as thyristors, Gate turn-off thyristors (GTO), Super-GTO, four layer (pnpn) diodes commonly referred to as Shockley diodes, breakover diodes (BOD), reverse switching rectifiers (RSR), or reverse blocking diode thyristors (RBDT), light activated silicon switches, IGBT, MCT, current or voltage controlled solidtrons, and avalanche transistors in various implementations. In other implementations, the switches 720a, 720b, 720c, 720d may be non-semiconductor based switches such as spark gaps and vacuum switches. The semiconductor material in the semiconductor switches may be silicon based in some implementations or other semiconductor materials such as GaAs, SiC, or GaN.

The shock wave generator 120 may apply the shock wave to the liquid based processing stream in a continuous flow process. Various configurations of the load 750 are suitable. An illustrative load 770 is shown in FIG. 6, in which the cathode 762 and the anode 764 of the pulsed electric field generator 700 included in shock wave generator 120 may be provided in a pipe 751, where the pipe 751 communicates the liquid based processing stream. The pipe 751, as illustrated, defines an inner surface 753 and an interior 754 with the liquid based processing stream passing thru the interior 754. The cathode 762 of the pulsed electric field generator 700 may be placed in the interior 754 of pipe 751, for example, generally along the centerline 755. The inner surface 753 functions as the anode 764 in the illustrated implementation so that an electric field is applied across the anode 764 and cathode 762 to generate shock waves. The cathode 762 and the anode 764 may be designed to shape the voltage pulse across the cell to provide generally a quasi-uniform shock wave between the cathode 762 and the anode 764. Upon study of the present disclosure, one of ordinary skill in the art would recognize that the anode 764 and cathode 762 may be interchanged without a significant change in form or function. In other variations, a plurality of anodes 764 and a plurality of cathodes 762 may be provided and may be generally disposed about the interior 754 of the pipe 751 circumferentially and/or axially in various ways in various implementations to provide shock waves generally to the liquid based processing stream passing through the interior 754 of the pipe 751. A plurality of pulsed electric field generators 700 is in electrical communication with the plurality of cathodes 762 to generate the shockwaves in some variations. In addition a plurality of pulsed electric field generators 700 can be in electrical communication with a plurality of loads 770. In various implementations, the pipe 751 may be rectangular, square, or other cross-sectional shape. In some implementations, the shock wave may be applied to the liquid based processing stream in batch mode in ways that would be recognized by persons of ordinary skill in the art upon study of this disclosure.

Methods described herein may include, in some aspects, providing a ethanol production facility apparatus 10 using grain as feedstock and having a liquid based processing stream, the ethanol production facility apparatus 10 including at least one shock wave generator. The methods may include applying a shock wave to the liquid based processing stream at power and frequency effective to cause the disruption of starch from other portions of the grain including fiber, protein, and lipids using the at least one shock wave generator, and may include applying a shock wave to the liquid based processing stream at power and frequency effective to cause generally the dissolution of starch microcrystalline structures. In various aspects, the methods may include generating shock waves in the liquid based processing stream at pressure and frequency effective to denature starch molecules using the shock wave generator. In various aspects, the methods may include generating shock waves in the liquid based processing stream at pressure and frequency effective to cleave starch molecules using the shock wave generator.

The methods may include including a mill 510, cooker 520, a fermenter 550 in the ethanol production facility apparatus 10 and applying the shock wave to the liquid based processing stream between the mill 510 and the cooker 520, applying the shock wave to the liquid based processing stream in conjunction with the cooker 520, applying the shock wave to the liquid based processing stream between the cooker 520 and the fermenter 550 to dissolve generally starch microcrystalline structures using one or more shock wave generators, in various aspects. In various aspects, the methods may include configuring the ethanol production facility apparatus 10 with a wet mill 600 and applying shock waves at one or more locations in the wet milling 600 configured to cause generally separation of starch from other portions of the grain and/or dissolution of starch microcrystalline structures using one or more shock wave generators.

The methods may, in some aspects, include providing an ethanol production facility apparatus 11 using cellulosic biomass as feedstock and having a liquid based processing stream, the ethanol production facility apparatus 11 including at least one shock wave generator. The methods may include applying a shock wave to the liquid based processing stream at power and frequency effective to cause the disruption of the cellulose and hemicellulose structures from lignin using the at least one shock wave generator. The methods may include including a milling unit 810, separation unit 815, and fermenter 850 in the ethanol production facility apparatus 11 and applying the shock wave to the liquid based processing stream between the milling unit 810 and the separation unit 815, applying the shock wave to the liquid based processing stream in conjunction with the separation unit 815, and applying the shock wave to the liquid based processing stream between the separation unit 815 and the fermenter 850 to cause the disruption of the cellulosic material structures from lignin using one or more shock wave generators, in various aspects.

In various aspects, the methods may include providing an ethanol production facility apparatus 1 having a liquid based processing stream, the ethanol production facility apparatus including at least one shock wave generator, and applying a shock wave to the liquid based processing stream at power and frequency effective to kill bacteria in order to control the bacteria level using the at least one shock wave generator. The methods may include applying the shock wave to the liquid based processing stream at a location effective to control the bacteria level in the fermenter 550 in the ethanol production facility apparatus 10 using grain as feedstock. The methods may include applying the shock wave to the liquid based processing stream at a location effective to control the bacteria level in the fermenter 850 in the ethanol production facility apparatus 11 using cellulosic biomass as feedstock.

The foregoing discussion discloses and describes merely exemplary implementations. Upon study of the specification, one of ordinary skill in the art will readily recognize from such discussion, and from the accompanying figures and claims, that various changes, modifications and variations can be made therein without departing from the spirit and scope of the invention as defined in the following claims.

What is claimed is:

1. An ethanol production facility apparatus comprising:
a plurality of process units for converting feedstock into ethanol, the process units being in fluid communication to enable a liquid based processing stream to flow among the process units; and
a shock wave generator configured to introduce a shock wave into the liquid based processing stream.

2. The ethanol production facility apparatus, as in claim 1, wherein the shock wave generator is configured to introduce the shock wave in the liquid based processing stream between two of the process units.

3. The ethanol production facility apparatus, as in claim 1, wherein the shock wave generator is configured to introduce the shock wave in the liquid based processing stream within at least one of the process units.

4. The ethanol production facility apparatus, as in claim 1, wherein the liquid based processing stream comprises starch microcrystalline structures, the shock wave generator being configured to generate the shock wave at a power and frequency effective to cause generally dissolution of the starch microcrystalline structures.

5. The ethanol production facility apparatus, as in claim 1, wherein the liquid based processing stream comprises grain fragments, the shock wave generator being configured to generate the shock wave at a power and frequency effective to loosen generally the structure of the grain fragments.

6. The ethanol production facility apparatus, as in claim 1, wherein the liquid based processing stream comprises starch-containing grain fragments, the shock wave generator being configured to generate the shock wave at a power and frequency effective to cause generally the separation of the starch in the grain fragments from other portions of the grain fragments.

7. The ethanol production facility apparatus, as in claim 6, wherein the other portions of the grain fragments comprise fiber.

8. The ethanol production facility apparatus, as in claim 6, wherein the other portions of the grain fragments comprise protein.

9. The ethanol production facility apparatus, as in claim 6, wherein the other portions of the grain fragments comprise lipids.

10. The ethanol production facility apparatus, as in claim 1, wherein the liquid based processing stream comprises starch molecules, the shock wave generator being configured to generate the shock wave at a power and frequency effective to denature the starch molecules.

11. The ethanol production facility apparatus, as in claim 1, wherein the liquid based processing stream comprises starch molecules, the shock wave generator being configured to generate the shock wave at a power and frequency effective to cleave the starch molecules.

12. The ethanol production facility apparatus, as in claim 1, wherein the liquid based processing stream comprises cellulosic biomass fragments containing cellulosic material and lignin, the shock wave generator being configured to generate the shock wave at a power and frequency effective to enhance separation of the cellulosic material from the lignin.

13. The ethanol production facility apparatus, as in claim 1, wherein the liquid based processing stream contains cellulosic material, the shock wave generator being configured to generate the shock wave at a power and frequency effective to hydrolyze cellulosic material.

14. The ethanol production facility apparatus, as in claim 1, wherein the liquid based processing stream comprises bacteria, the shock wave generator being configured to generate the shock wave at a power and frequency effective to generally kill the bacteria in the liquid based processing stream.

15. The ethanol production facility apparatus, as in claim 14, wherein at least one of the process units is configured as a fermenter, the shock wave generator being configured to generate the shock wave in the liquid based processing stream at a location upstream of the fermenter to control bacteria level in the fermenter.

16. The ethanol production facility apparatus, as in claim 1, wherein the shock wave generator comprises one or more semiconductor switches.

17. An ethanol production facility apparatus comprising:
a mill;
a first flow conduit;
a cooker in fluid communication with the mill through the first flow conduit;
a first shock wave generator for introducing into the first flow conduit a shock wave configured to dissolve starch microcrystalline structures in a liquid-based ethanol processing stream;
a second shock wave generator for introducing into the cooker a shock wave configured to dissolve starch microcrystalline structures in a liquid-based ethanol processing stream;
a second flow conduit;
a fermenter unit in fluid communication with the cooker through the second flow conduit; and
a third shock wave generator for introducing into the second flow conduit a shock wave configured to generally kill bacteria in a liquid-based ethanol processing stream.

18. An ethanol production facility apparatus comprising:
a milling unit;
a first flow conduit;
a separation unit in fluid communication with the milling unit through the first flow conduit;
a first shock wave generator for introducing into the first flow conduit a shock wave configured to separate cellulosic material from lignin in a liquid-based ethanol processing stream;
a second shock wave generator for introducing into the separation unit a shock wave configured to separate cellulosic material from lignin in a liquid-based ethanol processing stream;
a second flow conduit;
a liquefaction unit in fluid communication with the separation unit through the second flow conduit;
a third shock wave generator for introducing into the second flow conduit a shock wave configured to separate cellulosic material from lignin in a liquid-based ethanol processing stream;
a third flow conduit;
a fermenter in fluid communication with the liquefaction unit through the third flow conduit;
a fourth shock wave generator for introducing into the third flow conduit a shock wave generally kill bacteria in a liquid-based ethanol processing stream.

19. An ethanol production facility apparatus, comprising:
means for processing a liquid based processing stream to produce ethanol; and
means for generating a shock wave in the liquid based processing stream.

20. A method of obtaining ethanol from feedstock, comprising:
producing a liquid based processing stream from the feedstock;
introducing a shock wave into the liquid based processing stream to condition the liquid based processing stream for ethanol production; and
processing the liquid based processing stream in a plurality of process units to obtain the ethanol.

21. The method, as in claim 20, wherein the introducing step comprises:
introducing the shock wave in the liquid based processing stream within at least one of the process units.

22. The method, as in claim 20, wherein the introducing step comprises:
introducing the shock wave in the liquid based processing stream at a location between at least two of the process units.

23. The method, as in claim 20, wherein the liquid based processing stream includes starch microcrystalline structures, further comprising:
generating the shock wave with a shock wave generator at a power and frequency effective to cause generally dissolution of the starch microcrystalline structures.

24. The method, as in claim 20, wherein the liquid based processing stream includes starch molecules, further comprising:
generating the shock wave with a shock wave generator at a power and frequency effective generally to denature the starch molecules.

25. The method, as in claim 20, wherein the liquid based processing stream includes starch molecules, further comprising:
generating the shock wave with a shock wave generator at a power and frequency effective to generally cleave the starch molecules.

26. The method, as in claim 20, wherein the liquid based processing stream includes grain fragments, further comprising:
generating the shock wave with a shock wave generator at a power and frequency effective to loosen generally structure of the grain fragments.

27. The method, as in claim 20, wherein the liquid based processing stream includes starch-containing grain fragments, further comprising:
generating the shock wave with a shock wave generator at a power and frequency effective to cause generally the separation of the starch in the grain fragments from other portions of the grain fragments.

28. The method, as in claim 27, wherein the other portions of the grain fragments comprise fiber.

29. The method, as in claim 27, wherein the other portions of the grain fragments comprise protein.

30. The method, as in claim 27, wherein the other portions of the grain fragments comprise lipids.

31. The method, as in claim 20, wherein the liquid based processing stream includes cellulosic biomass fragments containing cellulosic material and lignin, further comprising:
generating the shock wave with a shock wave generator at a power and frequency effective to enhance separation of the cellulosic material from the lignin.

32. The method, as in claim 20, wherein the liquid based processing stream includes bacteria, further comprising:
generating the shock wave with a shock wave generator at a power and frequency effective to generally kill the bacteria.

33. The method, as in claim 32, wherein:
at least one of the process units is a fermenter; and
the introducing step comprises introducing the shock wave in the liquid based processing stream at a location upstream of the fermenter to control bacteria level in the fermenter.

34. The method, as in claim 20, wherein the liquid based processing stream includes cellulosic material, further comprising:
generating the shock wave with a shock wave generator at a power and frequency effective to hydrolize the cellulosic material.
